(19) United States
(12) Patent Application Publication
(10) Pub. No.: US 2011/0177139 A1
(43) Pub. Date: Jul. 21, 2011

(54) SOLID MICROSTRUCTURE THAT ENABLES MULTIPLE CONTROLLED RELEASE AND METHOD OF MAUFACTURING SAME

(75) Inventors: Hyungil Jung, Seoul (KR); Kwang Lee, Seoul (KR)

(73) Assignee: NURIM WELLNESS CO. LTD., Seoul (KR)

(21) Appl. No.: 13/122,391

(22) PCT Filed: Oct. 1, 2009
(86) PCT No.: PCT/KR09/05654

§ 371 Date: Apr. 1, 2011

(30) Foreign Application Priority Data
Oct. 1, 2008 (KR) 10-2008-0096699

(51) Int. Cl. A61K 9/00; A61K 47/34; A61K 47/32; A61K 47/36; A61K 47/38; A61K 47/12; A61K 47/26; B82Y 5/00

(52) U.S. Cl. 424/400; 514/772.3; 514/772.5; 514/772.4; 514/777; 514/781; 514/784; 514/779; 514/778; 977/773; 977/906

(57) ABSTRACT

Provided are a method of manufacturing a solid microstructure capable of controlling multidrug release by mixing a biocompatible or biodegradable material with microparticles or nanoparticles and/or an emulsion as drug carriers and a solid microstructure structure manufactured using the same.

### BACKGROUND

1. Field of the Invention

The present invention relates to a biocompatible/biodegradable solid microstructure capable of controlling multidrug release and a method of manufacturing the same.

2. Discussion of Related Art

While various drugs and therapeutic agents for treating diseases have been developed, in delivery into a human body, they still have aspects in need of improvement such as transmitting across biological barriers (e.g., skin, oral mucosa, blood-brain barriers, etc.) and efficiency in drug delivery.

Generally, drugs are orally administered in tablet or capsule formulation. However, since various drugs are digested or absorbed in the gastrointestinal tract or lost due to mechanisms occurring in the liver, these drugs cannot be effectively delivered by only such oral administration. In addition, some drugs cannot be effectively diffused through an intestinal mucosa. Furthermore, patient compliance becomes a problem (for example, in patients who need to take drugs at predetermined intervals, or critical patients who cannot take drugs).

Another common technique for drug delivery is using a conventional needle. This technique is more effective than oral administration, but may cause pain in an injected area, local damage to skin, bleeding, and infection in the injected area.

To solve these problems, various microstructures including a microneedle have been developed.

In general, a biodegradable solid microneedle has been used for the in vivo delivery of drugs. The delivery of drugs using the biodegradable solid microneedle is aimed not at the delivery of drugs to a bio-circulatory system, such as a blood vessel or lymphatic vessel, but at transdermal drug delivery. Accordingly, the biodegradable solid microneedle needs to pierce the skin without pain and be mixed with drugs during a manufacturing process and dissolved in a living body after the piercing of the skin to enable the delivery of the drugs to a target region. In addition, the biodegradable microneedle needs to have such sufficient physical hardness as to pierce the 10 to 20 μm-thick stratum corneum of the skin.

In 2005, an absorbable microneedle was manufactured by Nano Device and Systems Inc. (Japanese Patent Publication No. 2005154321; and “Sugar Micro Needles as Transdermic Drug Delivery System,” Biomedical Microdevices 7:3, 185-188, 2005). Such an absorbable microneedle is used in drug delivery or cosmetics without removal of the microneedle inserted intradermally. According to the above-mentioned method, a composition prepared by mixing maltose and a drug was applied to a template and then solidified so as to manufacture a microneedle. Meanwhile, Prausnitz (University of Georgia, U.S.A.) suggested a method of manufacturing a biodegradable polymer microneedle by forming a template by performing etching or photolithography on glass (Biodegradable Polymer Microneedles: Fabrication, Mechanics and Transdermal Drug Delivery, Journal of Controlled Release 104, 51-66, 2005). The above-described patents disclose that an absorbable microneedle for transdermal absorption of drugs is manufactured. However, the transdermal delivery of the drugs was accompanied by pain. In addition, due to a technical limit in the manufacture of a template, it is impossible to manufacture a microneedle whose top has a suitable diameter to achieve the painless absorption and which has a length required for effective drug delivery, that is, a length of 1 mm or more. Also, the above-described patents disclose a microneedle capable of mounting only chemical drugs according to a high manufacturing temperature.

Furthermore, Prausnitz (University of Georgia, U.S.A.) proposed a method of manufacturing a multilayered biodegradable microneedle (International Patent Application No. WO02/064193; and “Polymer Particle-based Micromolding to Fabricate Novel Microstructure” BiomedicalMicrodevices 9:223-234, 2007). The method includes melting a portion of a microneedle, which is manufactured by injecting microparticles formed of a biodegradable plastic into a template and melting the microparticles by pressing, and melting portions of the microparticles by pressing the portions of the microneedle and the microparticles against the template to manufacture a multilayered microneedle. However, since the multilayered microneedle is weaker than a single microneedle, it is difficult to effectively separate the multilayered microneedle from the template and the multilayered microneedle is likely to break during skin penetration. In addition, due to a technical limit in the manufacture of a template, only one type of encapsulated biodegradable microneedle can be manufactured. Accordingly, it is impossible to manufacture various types of biodegradable microneedles capable of controlling multidrug release, thus precluding satisfaction of diversity in drug delivery.

Accordingly, it is necessary to develop a method of manufacturing a biodegradable microneedle using both chemical and biological drugs and a biodegradable microneedle capable of controlling drug release to maximize the efficiency of drug delivery. Ultimately, there are ongoing demands for a biodegradable solid microneedle capable of controlling the release of each of a plurality of drugs.

A plurality of papers and patent documents are referred to as cited references throughout the present specification, the disclosure of which is incorporated herein by reference in its entirety to describe the technical scope of the present invention more apparently.

### SUMMARY OF THE INVENTION

The present inventors have made efforts to develop a solid microstructure having a microscale diameter and sufficient effective length and hardness that is capable of controlling multidrug release after proposing a biodegradable solid microstructure with an advanced outward shape and hardness using a drawing method (PCT Application No. PCT/KR2007/003506: A Biodegradable Solid-type Microneedle and Methods for Preparing it). As a result, they have found that a solid microstructure manufactured by mixing a biocompatible material or biodegradable material with microparticles, nanoparticles, and/or an emulsion as drug carriers had the above-described features, thereby completing the present invention.

Thus, the present invention is directed to a method of manufacturing a solid microstructure that contains a mixture of water-soluble and fat-soluble drugs, cosmetic ingredients, or polymers as a multidrug, enables a multidrug release function, and has desired properties, for example, desired effective length, top diameter, and hardness.

The present invention is also directed to a solid microstructure manufactured using the same.

The foregoing and other objects, features, and advantages of the invention will be more apparent from the detailed description of exemplary embodiments of the invention and the appended claims with reference to the accompanying drawings.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIGS. 1A and 1B are diagrams illustrating a configuration of a solid microneedle for drug release manufactured by mixing a biocompatible or biodegradable material alone or a biocompatible or biodegradable material containing a fat-soluble drug with a water-soluble drug using a water-in-oil (W/O) emulsion method according to an exemplary embodiment of the present invention;

FIGS. 2A and 2B are diagrams illustrating a configuration of a solid microneedle capable of controlling multidrug release manufactured by mixing a biocompatible or biodegradable material alone or a biocompatible or biodegradable material containing a water-soluble drug with a fat-soluble drug using an oil-in-water (O/W) emulsion method according to an exemplary embodiment of the present invention;

FIGS. 3A and 3B are diagrams illustrating a configuration of a solid microneedle capable of multidrug release manufactured by secondarily emulsifying and mixing a biocompatible or biodegradable material alone or a biocompatible or biodegradable material containing a water-soluble drug in and with a W/O emulsion solution containing a fat-soluble drug and a water-soluble drug using a water-in-oil-in-water (W/O/W) emulsion method according to an exemplary embodiment of the present invention;

FIGS. 4A and 4B are diagrams illustrating a configuration of a solid microneedle for multidrug release manufactured by secondarily emulsifying and mixing a biocompatible or biodegradable material alone or a biocompatible or biodegradable material containing a fat-soluble drug in and with an O/W solution containing a water-soluble drug and a fat-soluble drug using an oil-in-water-in-oil (O/W/O) emulsion method according to an exemplary embodiment of the present invention;

FIGS. 5A through 5C are diagrams illustrating a process of controlling multidrug release by simultaneously delivering or sustainedly releasing water-soluble and fat-soluble drugs when the solid microstructures shown in FIG. 1, 2, 3, or 4 are manufactured in a patch form and applied to the skin;

FIGS. 6A and 6B are diagrams illustrating a configuration of a solid microneedle capable of controlling multidrug release manufactured by mixing a biocompatible or biodegradable material alone or a biocompatible or biodegradable material containing a water-soluble drug with microparticles or nanoparticles containing the same drug or a different drug, according to an exemplary embodiment of the present invention, wherein when the microparticles or nanoparticles contain the same drug, the drug release may be controlled at a desired time;

FIGS. 7A and 7B are diagrams illustrating a configuration of a solid microneedle capable of controlling multidrug release manufactured by mixing a biocompatible or biodegradable material alone or a biocompatible or biodegradable material containing a fat-soluble drug with microparticles or nanoparticles containing the same drug or a different drug, according to an exemplary embodiment of the present invention, wherein when the microparticles or nanoparticles contain the same drug, the drug release may be controlled at a desired time;

FIGS. 8A and 8B are diagrams illustrating a configuration of a solid microneedle capable of controlling multidrug release manufactured by mixing a biocompatible or biodegradable material alone or a biocompatible or biodegradable material containing a water-soluble drug with a fat-soluble drug using an O/W emulsion method and mixing the mixture with microparticles or nanoparticles containing another drug according to an exemplary embodiment of the present invention;

FIGS. 9A and 9B are diagrams illustrating a configuration of a solid microneedle for drug release manufactured by mixing a biocompatible or biodegradable material alone or a biocompatible or biodegradable material containing a water-soluble drug with a fat-soluble drug and a water-soluble drug using a W/O emulsion method, secondarily emulsifying the mixture using a W/O/W emulsion method, and mixing the W/O/W emulsion mixture with microparticles or nanoparticles containing another drug, according to an exemplary embodiment of the present invention;

FIGS. 10A, 10B and 10C are diagrams illustrating a process of controlling multidrug release when the solid microstructures shown in FIG. 6, 7, 8, or 9 are manufactured in a patch form and applied to the skin, wherein FIG. 10A shows that the biocompatible or biodegradable material starts to decompose when the solid microneedles are applied to the skin, FIG. 10B shows that the water-soluble and fat-soluble drugs are simultaneously delivered, and FIG. 10C shows that the drug contained in the microparticles or nanoparticles is finally released;

FIG. 11 is a graph showing a release profile of calcein in the microneedle containing the calcein-incorporated emulsion of Example 1; and

FIG. 12 is a graph showing a release profile of a maltose biodegradable microneedle (on which Cy5.5 is mounted) containing microparticles (on which calcein is mounted).

### DETAILED DESCRIPTION OF EXEMPLARY EMBODIMENTS

Exemplary embodiments of the present invention will be described in detail below with reference to the accompanying drawings. While the present invention is shown and described in connection with exemplary embodiments thereof, it will be apparent to those skilled in the art that various modifications can be made without departing from the spirit and scope of the invention.

According to an aspect of the present invention, there is provided a method of manufacturing a solid microstructure including: (a) preparing a biocompatible or biodegradable material as a scaffold material of the solid microstructure; (b) mixing the biocompatible or biodegradable material of step (a) with a drug that is incorporated in microparticles, nanoparticles, or an emulsion; and (c) manufacturing a solid microstructure containing drug carriers using the mixture of step (b).

According to another aspect of the present invention, there is provided a solid microstructure manufactured using the above-described method of the present invention.

The present inventors have made efforts to develop a solid microstructure having a microscale diameter and a sufficient effective length and hardness that is capable of controlling multidrug release. As a result, by mixing microparticles or nanoparticles and/or an emulsion as drug carriers with a biocompatible or biodegradable material, a new solid microstructure having the above-described merits was developed. According to the present invention, it was identified that a solid microstructure manufactured according to the present invention could control a release speed of a drug, which could not be conventionally obtained, according to time, or further contain a mixture of water-soluble and fat-soluble drugs, a mixture of additional cosmetic ingredients or polymers as multidrugs, or have desired properties, for example, desired effective length, top diameter, and hardness.

Respective steps of the method of the present invention will be described in detail as follows;

Step (a): Preparation of Biocompatible Material or Biodegradable Material as Scaffold Material of Solid Microstructure

A material used to manufacture a solid microstructure in the present invention may be a biocompatible material or a biodegradable material. In the present specification, the term “biocompatible material” refers to a material that is substantially non-toxic to the human body, chemically inactive, and devoid of immunogenicity. In the present specification, the term “biodegradable material” refers to a material that is degradable by body fluids or microorganisms in living bodies.

The biocompatible material and/or biodegradable material used in the present invention may form a scaffold material of the microstructure and be preferably, polyhydroxyalkanoates (PHAs), poly(α-hydroxy acid), poly(β-hydroxy acid), poly(3-hydroxybutyrate-co-valerate) (PHBV), poly(3-hydroxy proprionate) (PHP), poly(3-hydroxyhexanoate) (PHH), poly(4-hydroxy acid), poly(4-hydroxybutyrate), poly(4-hydroxyvalerate), poly(4-hydroxyhexanoate), poly(ester amide), poly(ε-caprolactone) (PCL), polylactide (PLA), polyglycolide (PGA), poly(lactide-co-glycolide) (PLGA), polydioxanone, poly(ortho ester), polyetherester, polyanhydride, poly(glycolic acid-co-trimethylene carbonate), polyphosphoester (PPE), PPE urethane, poly(amino acid), polycyanoacrylate, poly(trimethylene carbonate), poly(iminocarbonate), poly(tyrosine carbonate), polycarbonate (PC), poly(tyrosine arylate), polyalkylene oxalate, polyphosphazene, PHA-PEG, polyvinylpyrrolidone (PVP), polybutadiene, polyhydroxy butyric acid, polymethyl methacrylate, polymethacrylic acid ester, polypropylene, polystyrene, polyvinylacetal diethylamino acetate, polyvinyl acetate, polyvinyl alcohol (PVA), polyvinyl butyral, polyvinyl formal, vinyl chloride-propylene-vinyl acetate copolymer, vinyl chloride-vinyl acetate copolymer, coumarone-indene polymer, dibutylamino hydroxypropyl ether, ethylene-vinyl acetate copolymer, glycerol distearate, 2-methyl-5-vinyl pyridine methacrylate-methacrylic acid copolymer, hyaluronic acid, myristic acid, palmitic acid, stearic acid, benenic acid, cellulose or derivatives thereof, maltose, dextran, glucomannan, glucosamine, chitosan, heparin, alginate, inulin, starch, glycogen, chitin, chondroitin, dextrin, keratan sulfate, beef tallow, whale wax, beeswax, paraffin wax, or castor wax. More preferably, the biocompatible material and/or biodegradable material may be PLA, PGA, PLGA, cellulose or derivatives thereof, maltose, dextran, glucomannan, glucosamine, chitosan, heparin, alginate, inulin, starch, or glycogen. Most preferably, the biocompatible material and/or biodegradable material may be PLA, PGA, PLGA, cellulose or derivatives thereof, maltose, or chitosan. When a cellulose derivative is used as the biocompatible or biodegradable material, the cellulose derivative is preferably cellulose polymer, more preferably hydroxypropyl methylcellulose, hydroxyalkyl cellulose (preferably, hydroxyethyl cellulose or hydroxypropyl cellulose), ethyl hydroxyethyl cellulose, alkyl cellulose, or carboxymethylcellulose, still more preferably hydroxypropyl methylcellulose or carboxymethylcellulose, and most preferably carboxymethylcellulose.

The biocompatible or biodegradable material serving as the scaffold material of the microstructure may have self-viscosity or include other viscosity modifying agents. The viscosity of the biocompatible or biodegradable material may be variously changed according to the kinds, concentrations, and temperatures of materials contained therein or depending on whether or not a viscosity modifying agent is added and may be appropriately controlled according to the objects of the present invention.

For example, the viscosity of the biocompatible material or biodegradable material may be appropriately controlled by adding a viscosity modifying agent typically known to those skilled in the art, such as hyaluronic acid and salts thereof, PVP, cellulose polymer, dextran, gelatin, glycerin, polyethylene glycol, polysorbate, propylene glycol, povidone, carbomer, gum ghatti, guar gum, glucomannan, glucosamine, dammer resin, rennet casein, locust bean gum, microfibrillated cellulose, psyllium seedgum, xanthan gum, arabino galactan, gum arabic, alginic acid, gelatin, gellan gum, carrageenan, karaya gum, curdlan, chitosan, chitin, tara gum, tamarind gum, tragacanthgum, furcelleran, pectin, or pullulan to a principal component of a solid microstructure, for example, a biocompatible material or a biodegradable material.

According to an exemplary embodiment of the present invention, the biocompatible material or biodegradable material used in the present invention may be dissolved in an appropriate solvent to exhibit viscosity.

The solvent used to prepare the biocompatible material or biodegradable material is not specifically limited and may be selected from the group consisting of water, an absolute or hydrous lower alcohol having 1 to 4 carbon atoms, acetone, ethyl acetate, chloroform, dichloromethane, 1,3-butylene glycol, hexane, diethyl ether, and butylacetate.

Step (b): Mixture of Biocompatible or Biodegradable Material with Drug

Thereafter, the biocompatible or biodegradable material may be mixed with a drug, which may be incorporated in microparticles, nanoparticles, or an emulsion.

In the present specification, the term “microparticles” refers to microscale spheres configured to encapsulate the drug, and the term “nanoparticles” refers to nanoscale particles configured to encapsulate the drug. In the present specification, the term “emulsion” refers to the emulsification of the drug in the biocompatible or biodegradable material serving as the scaffold material of the solid microstructure.

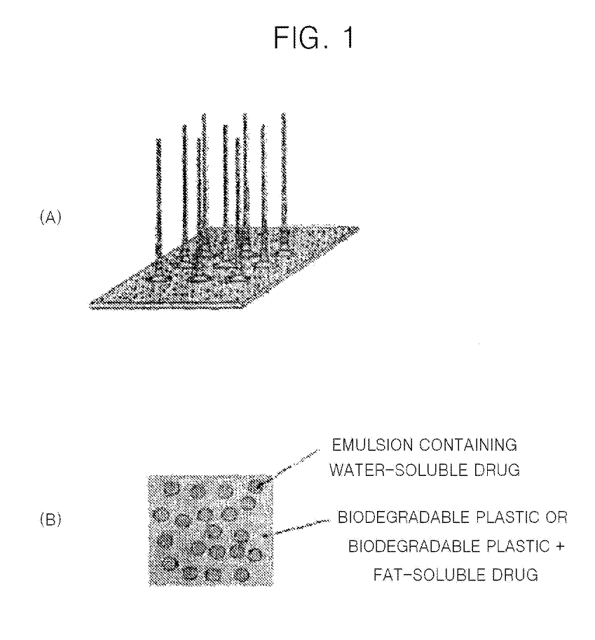

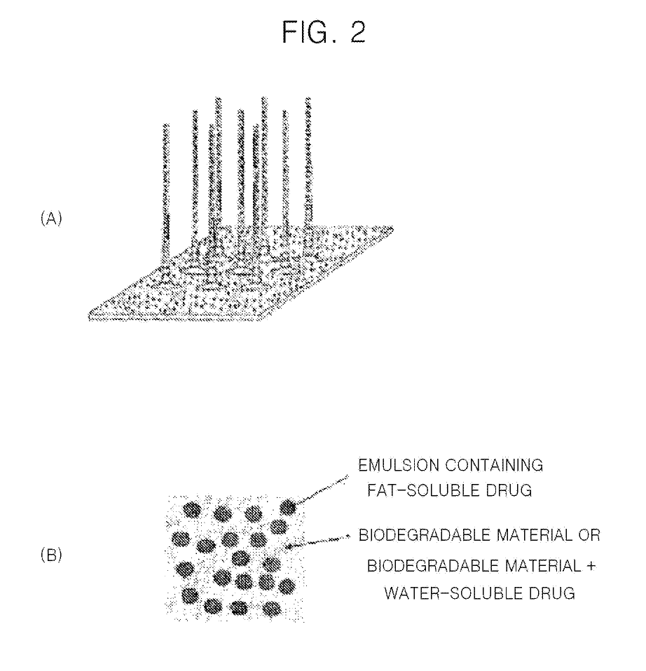

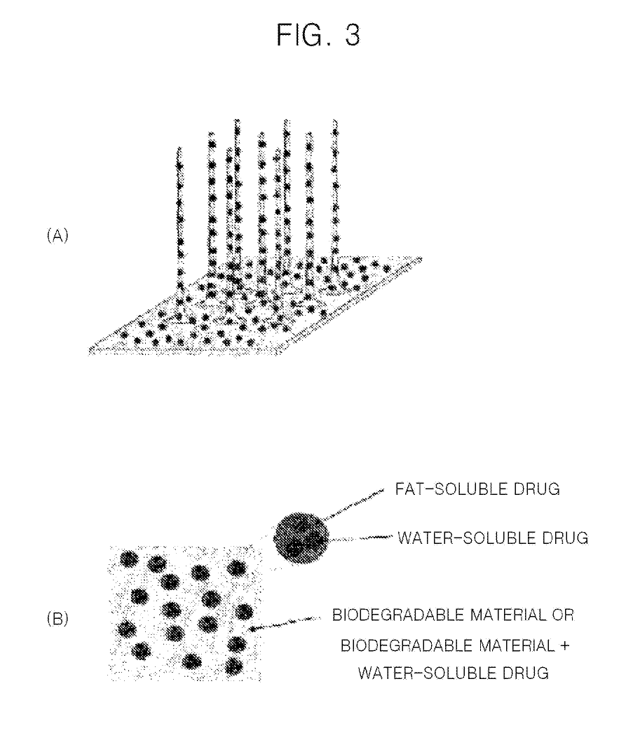

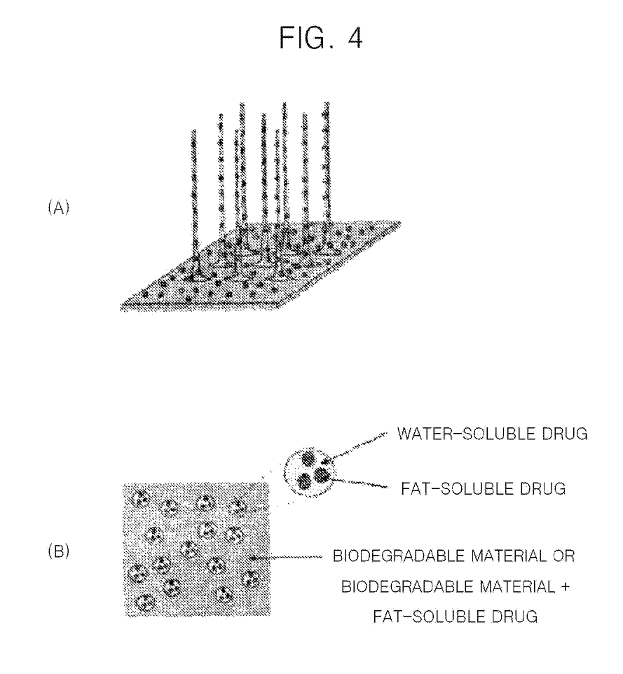

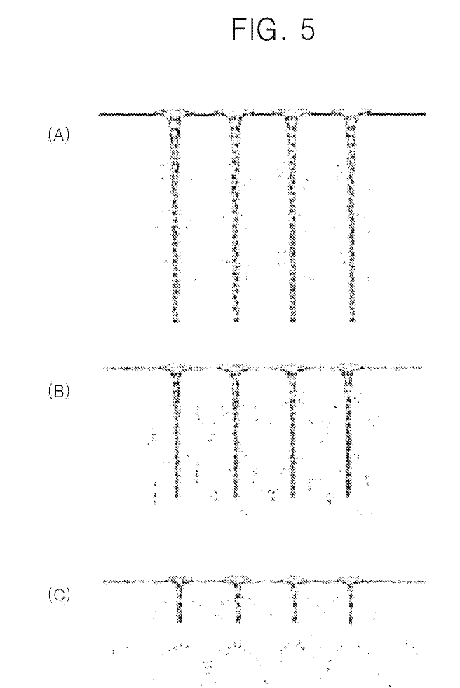

The biocompatible or biodegradable material serving as the scaffold material of the solid microstructure may be mixed with the incorporated drug using various methods. Three typical exemplary embodiments of the above methods will now be described as follows: According to a first exemplary embodiment, the biocompatible or biodegradable material may be mixed with only an emulsion (refer to FIGS. 1 through 4). The emulsion may be prepared using various methods known in the art by emulsifying a drug in a biocompatible or biodegradable material serving as a scaffold material of the solid microstructure. More specifically, the emulsion may be prepared in an oil-in-water (O/W) emulsion form, a water-in-oil (W/O) emulsion form, or a multiple emulsion form. Preferably, the preparation of the emulsion may include directly dispersing a drug in a biocompatible or biodegradable material in the absence of an emulsifier or incorporating a drug using various natural or synthetic emulsifiers. When the emulsion is prepared using an emulsifier, the emulsion may be more preferably stabilized using one natural emulsifier, such as lecithin, borax, stearic acid, amisol soft, helio gel, beeswax, xanthan gum, emulsifying wax, or a solubilizer. Alternatively, a drug-containing emulsion may be prepared using at least one synthetic emulsifier selected from the group consisting of O/W emulsifiers including PEG-8 dilaurate, PEG-150 distearate, PEG-8 stearate, PEG-40 distearate, and PEG-100 distearate and W/O emulsifiers including sorbitan stearate, sorbitan oleate, sorbitan sesquioleate, and sorbitan trioleate or a combination thereof. Most preferably, the emulsion may be prepared in the absence of an emulsifier. For example, a fat-soluble drug may be emulsified in a W/O form in a water-soluble biocompatible or biodegradable material using a homogenizer, thereby preparing a mixture. Alternatively, a water-soluble drug may be emulsified in an O/W form in a fat-soluble biocompatible or biodegradable material, such as chitosan or biodegradable plastic, using a homogenizer, thereby preparing a mixture. In addition, a W/O emulsion solution containing a fat-soluble drug and a water-soluble drug may be secondarily emulsified in a water-in-oil-in-water (W/O/W) form in a mixture of a water-soluble biocompatible or biodegradable material and a water-soluble drug using a multiple emulsion method, thereby preparing the mixture. Alternatively, an O/W emulsion solution containing a water-soluble drug and a fat-soluble drug may be secondarily emulsified in an oil-in-water-in-oil (O/W/O) form in a mixture of a fat-soluble biocompatible/biodegradable material and a fat-soluble drug, thereby preparing the mixture. The size of the prepared emulsion of the drug may be varied according to a speed with which the emulsion is homogenized.

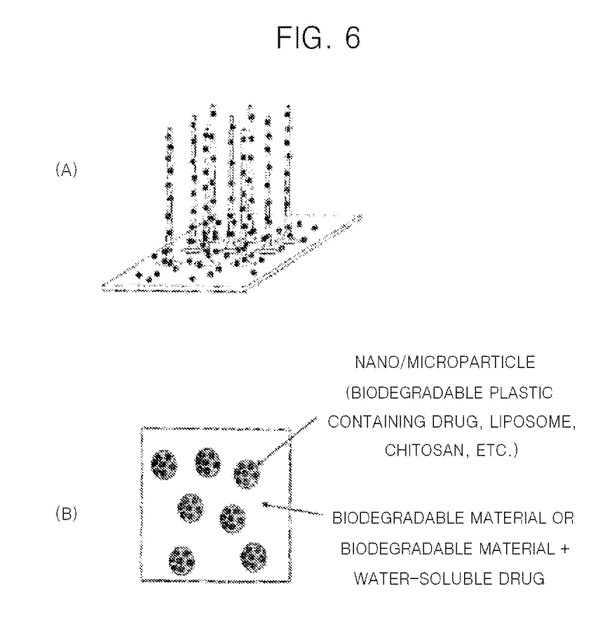

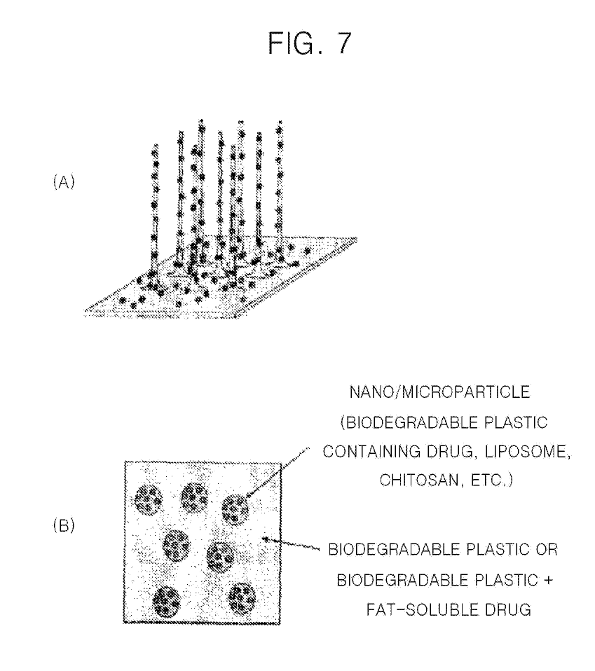

According to a second exemplary embodiment, the biocompatible material or biodegradable material may be mixed with microparticles or nanoparticles as drug carriers (refer to FIGS. 6 and 7). A method of mixing the biocompatible or biodegradable material with the microparticles or nanoparticles may be performed using various methods known in the art. For example, the biocompatible or biodegradable materials may be mixed with the microparticles or nanoparticles using a multiple emulsion method, a dispersion dry method, or a particle precipitation method.

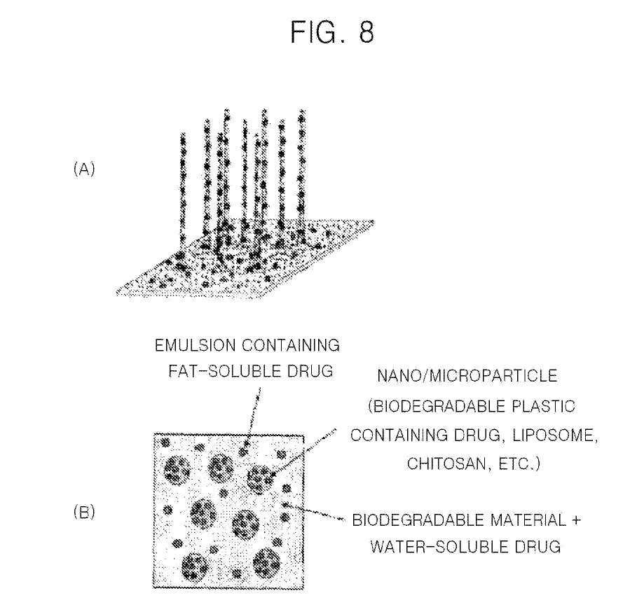

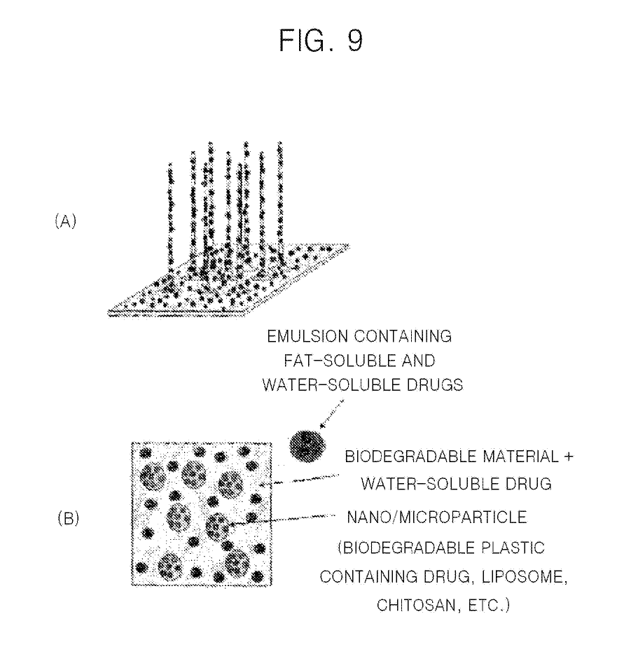

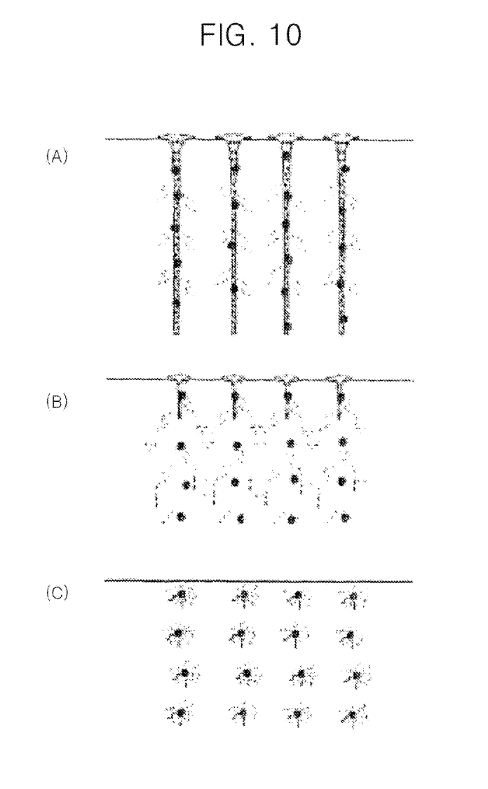

According to a third exemplary embodiment, the solid microstructure may contain microparticles or nanoparticles and emulsions, such as W/O emulsions, O/W emulsions, or multiple emulsions, as drug carriers (refer to FIGS. 8 and 9).

The drug mixed with the biocompatible or biodegradable material is not specifically limited. For example, the drug used in the present invention may include chemical drugs, protein medicines, peptide medicines, hexane molecules for gene therapy, and nanoparticles.

The drug used in the present invention may include, for example, anti-inflammatory agents, anodynes, antiarthritics, spasmolytics, antidepressants, antipsychotic drugs, tranquilizers, antianxiety drugs, narcotic antagonists, anti-Parkinson's disease drugs, cholinergic agonists, anticancer drugs, angiogenesis inhibitors, immunosupressants, antiviral agents, antibiotics, anorectic agents, anticholinergics, antihistamines, antimigraine drugs, hormone drugs, coronary, cerebrovascular, or peripheral vascular vasodilators, contraceptives, antithrombotic drugs, diuretics, antihypertensive drugs, remedies for cardiovascular diseases, or cosmetic ingredients (e.g., a wrinkle enhancer, a skin-aging inhibitor, or skin whitener), but the present invention is not limited thereto.

The protein medicines or peptide medicines used in the present invention are not specifically limited and may include hormones, hormone analogues, enzymes, enzyme inhibitors, signal transduction proteins or portions thereof, antibodies or portions thereof, single-chain antibodies, binding proteins or binding domains thereof, antigens, adhesion proteins, structural proteins, regulatory proteins, toxin proteins, cytokines, transcription factors, blood coagulation factors, or vaccines, but the present invention is not limited thereto. More specifically, the protein and peptide medicines may include insulin, insulin-like growthfactor 1 (IGF-1), growth hormones, erythropoietin, granulocyte-colony-stimulating factors (G-CSFs), granulocyte/macrophage-colony-stimulating factors (GM-CSFs), interferon-α, interferon-β, interferon-γ, interleukin-1α and β, interleukin-3, interleukin-4, interleukin-6, interleukin-2, epidermal growth factors (EGFs), calcitonin, adrenocorticotropic hormone (ACTH), tumor necrosis factors (TNFs), atobisban, buserelin, cetrorelix, deslorelin, desmopressin, dynorphin A (1-13), elcatonin, eleidosin, eptifibatide, growth hormone releasing hormone-II (GHRH-II), gonadorelin, goserelin, histrelin, leuprorelin, lypressin, octreotide, oxytocin, pitressin, secretin, sincalide, terlipressin, thymopentin, thymosine α1, triptorelin, bivalirudin, carbetocin, cyclosporin, exedine, lanreotide, luteinizing-hormone-releasing hormone (LHRH), nafarelin, parathormone, pramlintide, enfuvirtide (T-20), thymalfasin, and ziconotide.

Preferably, the microparticles or nanoparticles containing the drug may be prepared using polyester, PHAs, poly(α-hydroxy acid), poly(β-hydroxy acid), PHBV, PHP, PHH, poly(4-hydroxy acid), poly(4-hydroxybutyrate), poly(4-hydroxyvalerate), poly(4-hydroxyhexanoate), poly(ester amide), PCL, PLA, PGA, PLGA, polydioxanone, poly(ortho ester), polyetherester, polyanhydride, poly(glycolic acid-co-trimethylene carbonate), PPE, PPE urethane, poly(amino acid), polycyanoacrylate, poly(trimethylene carbonate), poly(imino carbonate), poly(tyrosine carbonate), PC, poly(tyrosine arylate), polyalkylene oxalate, polyphosphazene, PHA-PEG, PVP, polybutadiene, polyhydroxy butyric acid, polymethyl methacrylate, polymethacrylic acid ester, polypropylene, polystyrene, polyvinylacetal diethylamino acetate, polyvinyl acetate, PVA, polyvinyl butyral, polyvinyl formal, vinyl chloride-propylene-vinyl acetate copolymer, vinyl chloride-vinyl acetate copolymer, coumarone-indene polymer, dibutylamino hydroxypropyl ether, ethylene-vinyl acetate copolymer, glycerol distearate, 2-methyl-5-vinyl pyridine methacrylate-methacrylic acid copolymer, hyaluronic acid, myristic acid, palmitic acid, stearic acid, benenic acid, cellulose or derivatives thereof, maltose, dextran, glucomannan, glucosamine, chitosan, heparin, alginate, inulin, starch, glycogen, chitin, chondroitin, dextrin, keratan sulfate, beef tallow, whale wax, beeswax, paraffin wax, or castor wax. More preferably, the microparticles or nanoparticles containing the drug may be prepared using PLA, PGA, PLGA, cellulose or derivatives thereof, maltose, dextran, glucomannan, glucosamine, chitosan, heparin, alginate, inulin, starch, or glycogen. Most preferably, the microparticles or nanoparticles containing the drug may be prepared using PLA, PGA, PLGA, cellulose or derivatives thereof, maltose, or chitosan. When a cellulose derivative is used as the biocompatible or biodegradable material, the cellulose derivative is preferably cellulose polymer, more preferably hydroxypropyl methylcellulose, hydroxyalkyl cellulose (preferably, hydroxyethyl cellulose or hydroxypropyl cellulose), ethyl hydroxyethyl cellulose, alkyl cellulose, or carboxymethylcellulose, still more preferably hydroxypropyl methylcellulose or carboxymethylcellulose, and most preferably carboxymethylcellulose.

According to an exemplary embodiment of the present invention, a biocompatible or biodegradable material used as a scaffold material of microstructures in the present invention may be a different material from the microparticles or nanoparticles.

According to an exemplary embodiment of the present invention, a biocompatible or biodegradable material used as a scaffold material of microstructures in the present invention may further contain a drug. Although the drug included in the biocompatible or biodegradable material is not limited, the drug may be more preferably different from a drug included in microparticles, nanoparticles, or an emulsion. Most preferably, a skeleton of microstructures, microparticles or nanoparticles, and an emulsion may contain different kinds of drugs, respectively.

One major feature of the present invention is the preparation of microstructures capable of controlling multidrug release by mounting various drugs in the microstructures. In the present specification, the term “control of drug release” refers to a capability of differently controlling the delivery effects of several drugs in a living body as needed using the fact that the biocompatible or biodegradable material and the microparticles or nanoparticles are biodegraded in vivo in different amounts of time to different extents.

Another major feature of the present invention is the mounting of one drug in microstructures in various ways. In this case, microstructures may be manufactured, which may be controlled to release the same drug at desired different times on the principle that a scaffold material of microstructures, microparticles, and nanoparticles are degraded at different speeds.

Drugs used in the present invention are not specifically limited. Preferably, a drug included in microparticles or nanoparticles and a drug included in an emulsion may be released at different speeds. Most preferably, a drug included in the scaffold material of the microstructure and a drug included in microparticles, nanoparticles, or an emulsion may be released at different speeds.

Step (c): Manufacture of Solid Microstructure Containing Drug Carrier

A drug-containing or drug-free biocompatible material or biodegradable material may be mixed with the drug carriers, and a solid microstructure may be finally manufactured. A method of manufacturing the solid microstructure may be performed using various methods known in the art. For example, a mixture prepared by emulsifying a drug in a biocompatible or biodegradable material in an O/W form or W/O form, a mixture prepared by mixing a biocompatible or biodegradable material with microparticles or nanoparticles containing a drug, or a mixture prepared by emulsifying a drug in a biocompatible or biodegradable material containing another drug in an O/W form or W/O form and mixing the emulsion with microparticles or nanoparticles containing yet another drug may be prepared on the surface of a substrate. A substrate configured to accommodate the mixture of the drug carriers and the biocompatible or biodegradable material is not specifically limited and may be formed of a material such as a polymer, an organic chemical material, a metal, a ceramic material, or a semiconductor material. Subsequently, the surface of the mixture may be contacted with a support manufactured in a desired form and drawn and solidified so that the mixture can be structured to have a reduced diameter from the substrate toward a contact surface between the mixture and the support. In this case, by applying strength equal to or higher than tensile strength to the mixture by increasing the drawing speed or by cutting a specific region using a laser, a thin, elongate biocompatible or biodegradable solid microstructure may be manufactured. Drawing conditions and drawing speed may be appropriately controlled using a typical method known in the art, for example, according to the viscosity or adhesiveness of the biocompatible or biodegradable material, the drug carriers, a viscosity modifying agent, or a solvent containing the mixture.

Finally, an arbitrary region of the drawn mixture may be cut, thereby obtaining a final solid microstructure. The cutting operation may be performed in various methods known in the art, for example, a physical cutting method of increasing a drawing speed or applying strength equal to or higher than stress, or a laser cutting method.

The present invention may provide various microstructures. Preferably, a microstructure according to the present invention may be a microneedle, a microspike, a microblade, a microknife, a microfiber, a microprobe, a microbarb, a microarray, or a microelectrode, more preferably a microneedle, a microspike, a microblade, a microknife, a microfiber, a microprobe or a microbarb, and most preferably a solid microneedle.

A microneedle needs to have a sufficiently thin and elongate structure to pierce the skin without pain and minimize the sense of a foreign body after insertion. A solid microneedle manufactured according to the present invention may be manufactured to a desired diameter and length without limitation. According to an exemplary embodiment of the present invention, a top of the microstructure may preferably have a diameter of 1 to 100 μm, more preferably 2 to 50 μm, and most preferably 5 to 10 μm. Also, the microstructure according to the present invention may preferably have an effective length of 100 to 10,000 μm, more preferably 200 to 10,000 μm, still more preferably 300 to 8,000 μm, and most preferably 500 to 2,000 μm. The above-described solid microstructure according to the present invention may be manufactured in patch and roller forms.

In the present specification, the term “top” refers to one terminal portion of the microstructure with the minimum diameter. In the present specification, the term “effective length” refers to a vertical length from the top of the microstructure to the surface of the lifting support. In the present specification, the term “solid microneedle” refers to an integrally manufactured microneedle without a hollow.

According to another aspect of the present invention, there is provided a solid microneedle for drug release, which includes a biocompatible or biodegradable material as a scaffold material of the solid microstructure, in which at least one drug carrier selected from the group consisting of microparticles, nanoparticles and an emulsion containing a drug is incorporated.

Since the solid microstructure according to the present invention employs the biocompatible or biodegradable material, the microparticles, the nanoparticles, or the emulsion, a description of the same parts of the solid microneedle according to the another aspect as that of the one aspect will be omitted for clarity.

According to an exemplary embodiment of the present invention, a biocompatible material or biodegradable material used as a scaffold material of the microstructures may be one selected from the group consisting of polyester, PHAs, poly(α-hydroxy acid), poly(β-hydroxy acid), PHBV, PHP, PHH, poly(4-hydroxy acid), poly(4-hydroxybutyrate), poly(4-hydroxyvalerate), poly(4-hydroxyhexanoate), poly(ester amide), PCL, PLA, PGA, PLGA, polydioxanone, poly(ortho ester), polyetherester, polyanhydride, poly(glycolic acid-co-trimethylene carbonate), PPE, PPE urethane, poly(amino acid), polycyanoacrylate, poly(trimethylene carbonate), poly(iminocarbonate), poly(tyrosine carbonate), PC, poly(tyrosine arylate), polyalkylene oxalate, polyphosphazene, PHA-PEG, PVP, polybutadiene, polyhydroxy butyric acid, polymethyl methacrylate, polymethacrylic acid ester, polypropylene, polystyrene, polyvinylacetal diethylamino acetate, polyvinyl acetate, PVA, polyvinyl butyral, polyvinyl formal, vinyl chloride-propylene-vinyl acetate copolymer, vinyl chloride-vinyl acetate copolymer, coumarone-indene polymer, dibutylamino hydroxypropyl ether, ethylene-vinyl acetate copolymer, glycerol distearate, 2-methyl-5-vinyl pyridine methacrylate-methacrylic acid copolymer, hyaluronic acid, myristic acid, palmitic acid, stearic acid, benenic acid, cellulose or derivatives thereof, maltose, dextran, glucomannan, glucosamine, chitosan, heparin, alginate, inulin, starch, glycogen, chitin, chondroitin, dextrin, keratan sulfate, beef tallow, whale wax, beeswax, paraffin wax, and castor wax. More preferably, the biocompatible material or biodegradable material may be PLA, PGA, PLGA, cellulose or derivatives thereof, maltose, dextran, glucomannan, glucosamine, chitosan, heparin, alginate, inulin, starch, or glycogen. Most preferably, the biocompatible material or biodegradable material may be PLA, PGA, PLGA, cellulose or derivatives thereof, maltose, or chitosan.

According to an exemplary embodiment of the present invention, microparticles or nanoparticles containing the drug may be selected from the group consisting of polyester, PHAs, poly(α-hydroxy acid), poly(β-hydroxy acid), PHBV, PHP, PHH, poly(4-hydroxy acid), poly(4-hydroxybutyrate), poly(4-hydroxyvalerate), poly(4-hydroxyhexanoate), poly(ester amide), PCL, PLA, PGA, PLGA, polydioxanone, poly(ortho ester), polyetherester, polyanhydride, poly(glycolic acid-co-trimethylene carbonate), PPE, PPE urethane, poly(amino acid), polycyanoacrylate, poly(trimethylene carbonate), poly(imino carbonate), poly(tyrosine carbonate), PC, poly(tyrosine arylate), polyalkylene oxalate, polyphosphazene, PHA-PEG, PVP, polybutadiene, polyhydroxy butyric acid, polymethyl methacrylate, polymethacrylic acid ester, polypropylene, polystyrene, polyvinylacetal diethylamino acetate, polyvinyl acetate, PVA, polyvinyl butyral, polyvinyl formal, vinyl chloride-propylene-vinyl acetate copolymer, vinyl chloride-vinyl acetate copolymer, coumarone-indene polymer, dibutylamino hydroxypropyl ether, ethylene-vinyl acetate copolymer, glycerol distearate, 2-methyl-5-vinyl pyridine methacrylate-methacrylic acid copolymer, hyaluronic acid, myristic acid, palmitic acid, stearic acid, benenic acid, cellulose or derivatives thereof, maltose, dextran, glucomannan, glucosamine, chitosan, heparin, alginate, inulin, starch, glycogen, chitin, chondroitin, dextrin, keratan sulfate, beef tallow, whale wax, beeswax, paraffin wax, and castor wax. More preferably, the microparticles or nanoparticles containing the drug may be prepared using PLA, PGA, PLGA, cellulose or derivatives thereof, maltose, dextran, glucomannan, glucosamine, chitosan, heparin, alginate, inulin, starch, or glycogen. Most preferably, the microparticles or nanoparticles containing the drug may be prepared using PLA, PGA, PLGA, cellulose or derivatives thereof, maltose, or chitosan.

According to an exemplary embodiment of the present invention, a biocompatible or biodegradable material used as a scaffold material of solid microstructures in the present invention may be a different material from the microparticles or nanoparticles.

According to an exemplary embodiment of the present invention, a solid microstructure may contain both microparticles or nanoparticles and an emulsion as drug carriers.

According to an exemplary embodiment of the present invention, a biocompatible or biodegradable material used as a scaffold material of solid microstructures in the present invention may further contain a drug. Although the drug included in the biocompatible or biodegradable material is not limited, the drug may be more preferably different from a drug included in microparticles, nanoparticles, or an emulsion. Most preferably, a skeleton of solid microstructures, microparticles or nanoparticles, and an emulsion may contain different kinds of drugs, respectively.

According to an exemplary embodiment of the present invention, a drug included in microparticles or nanoparticles as a component of the present invention and a drug included in an emulsion may be released at different speeds. Most preferably, a drug included in the scaffold material of the microstructure and a drug included in microparticles, nanoparticles, or an emulsion may be released at different speeds.

The present invention will now be described more fully with reference to the accompanying drawings, in which exemplary embodiments of the invention are shown. The exemplary embodiments should be considered in a descriptive sense only and not for purposes of limitation. Therefore, it will be apparent to those skilled in the art that the scope of the invention is not limited by the exemplary embodiments.

### EXAMPLES

### Example 1

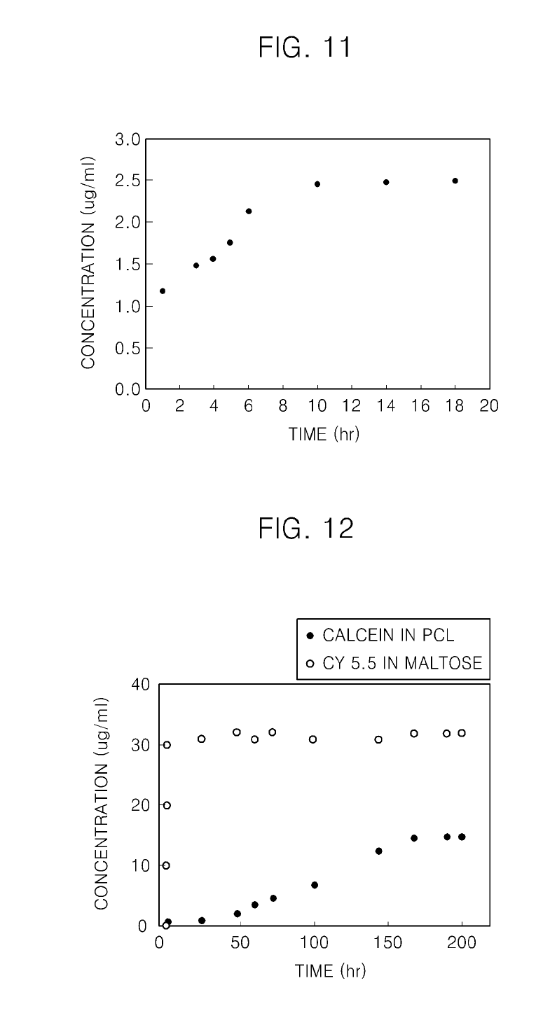

A biodegradable solid microneedle encapsulated in a W/O form was manufactured using poly-L-lactide (PLA) (Sigma) or poly(DL-lactide-co-glycolide) (PLGA) 50:50 as a biodegradable polymer. PLA or PLGA was dissolved in a dichloromethane (Sigma) solvent, mixed with a calcein (Sigma) solution, and emulsified in a W/O form using a homogenizer at a stirring speed of 24,000 rpm for 2 minutes. After the W/O-type emulsion solution was coated on a glass flat panel, a 3×3 patterned frame with a diameter of 200 μm was contacted with the glass flat panel. While solidifying the coated W/O-type emulsion solution due to strong volatility of dichloromethane, the frame was strongly contacted with the glass substrate. After 3 minutes, the coated W/O-type emulsion solution was drawn at a speed of 25 μm/s for 90 seconds using the frame contacted with the PLA or PLGA emulsion solution, thereby manufacturing a microneedle with a length of 2,200 μm. Subsequently, the manufactured solid microstructure was drawn at a higher speed or cut into and separated from the frame. By crystallizing the manufactured solid microstructure in a vacuum oven at a temperature of 140 or 170° C., an encapsulated biodegradable polymer microneedle with a top diameter of 5 μm and an effective length of 2,000 μm was manufactured. When a solid microneedle manufactured using a calcein-containing PLGA emulsion was impregnated with phosphate buffered saline (PBS), calcein was wholly released in 10 hours (refer to FIG. 11).

### Example 2

An O/W-type biodegradable microneedle was manufactured using carboxymethylcelluose (CMC) (Sigma) as a cellulose derivative. A water-soluble vitamin C derivative (ascorbic acid: Sigma) was mixed with water serving as a solvent, mixed with a fat-soluble vitamin A derivative (retinol: Sigma) dissolved in dichloromethane (Sigma) serving as an organic solvent, and emulsified in an O/W form using a homogenizer at a stirring speed of 11,000 rpm. Subsequently, CMC was dissolved in the emulsion solution, thereby producing a 4% CMC-containing O/W-type emulsion solution. After the CMC-containing O/W emulsion solution was coated on a glass flat panel to a predetermined thickness, a previously prepared 3×3 patterned frame with a diameter of 200 μm was contacted with the glass flat panel. Thereafter, the coated CMC emulsion surface was dried for 10 seconds to strongly contact the frame with CMC. The coated CMC O/W emulsion surface was drawn at a speed of 30 μm/s for 60 seconds using the frame contacted with CMC, thereby manufacturing a solid microstructure with an effective length of 1,800 μm. Subsequently, the solid microstructure was condensed and solidified using a strong dry process for 5 minutes and separated from the frame by increasing a drawing speed or cutting. As a result, a biodegradable cellulose microneedle in which vitamin A and vitamin C were encapsulated was manufactured to have a top diameter of 5 μm and an effective length of 1,800 μm. The particle size of the emulsion solution varied according to the stirring speed of the homogenizer, and the length of the manufactured solid microstructure varied according to the drawing speed and time.

### Example 3

A biodegradable microneedle containing encapsulated microparticles was manufactured using powdered maltose monohydrate (Sigma) as natural sugar. A maltose candy was prepared by melting the maltose powder at a temperature of 140° C. and mixed with Cy5.5 (Sigma). PCL (Aldrich) microparticles containing calcein (Sigma) were prepared through a multiple emulsion method using a homogenizer and filtered using a filter (Millex), thereby obtaining only microparticles with a diameter of 5 mm or less. The maltose candy mixture solution was mixed with the obtained microparticles, and a candy mixture of maltose and microparticles was coated to a predetermined thickness on a glass flat panel and contacted with a previously prepared 2×2 patterned frame with a diameter of 200 μm. Afterwards, the coated maltose candy was more strongly contacted with the frame for 10 seconds. The coated maltose candy was drawn at a speed of 30 μm/s for 60 seconds using the frame contacted with the maltose candy, thereby manufacturing a solid microneedle with an effective length of 1,800 μm. Subsequently, the manufactured solid microneedle could be separated from the frame by increasing a drawing speed or cutting. As a result, a biodegradable maltose microneedle containing encapsulated microparticles, which was capable of mounting multiple drugs and controlling multidrug release, was manufactured to have a top diameter of 5 μm and an effective length of 1,800 μm. A microneedle of the same type may be manufactured using CMC as a cellulose derivative instead of maltose. The size of microparticles varied according to a stirring speed of the homogenizer and depending on the presence of a filter used, and the length of a manufactured microneedle varied according to a drawing speed and drawing time. When a maltose biodegradable microneedle (on which Cy5.5 was mounted) containing microparticles (on which calcein was mounted) was impregnated with PBS, Cy5.5 contained in maltose was wholly released within 1 hour, and calcein contained in the microparticles was wholly released in 7 days, so that the maltose biodegradable microneedle could control the multidrug release of the multiple drugs (refer to FIG. 12).

### Example 4

A biodegradable microneedle capable of controlling multidrug release was manufactured using CMC through an O/W emulsion method and a microparticle encapsulation method. A water-soluble vitamin C derivative (ascorbic acid: Sigma) was mixed with water serving as a solvent, mixed with a fat-soluble vitamin A derivative (retinol: Sigma) dissolved in dichloromethane (Sigma) serving as an organic solvent, and emulsified in an O/W form using a homogenizer at a stirring speed of 11,000 rpm. Afterwards, CMC was dissolved in the emulsion solution, thereby producing a 4% CMC O/W emulsion solution. In addition, PLC (Sigma) microparticles containing calcein (Sigma) were prepared through a multiple emulsion method and filtered using a filter (Millex), thereby obtaining only microparticles with a diameter of 5 mm or less.

The CMC-containing O/W-type emulsion solution was mixed with the microparticles containing encapsulated calcein, and the resultant mixture solution was coated to a predetermined thickness on a glass flat panel and contacted with a previously prepared 2×2 patterned frame with a diameter of 200 μm. Afterwards, a coated surface of the CMC-containing emulsion containing the microparticles was dried for 10 seconds to strongly contact CMC with the frame. The coated surface of the CMC-containing O/W-type emulsion was drawn at a speed of 30 μm/s for 60 seconds using the frame contacted with CMC, thereby manufacturing a solid microneedle with an effective length of 1,800 μm. Subsequently, the manufactured solid microneedle was condensed and solidified using a strong dry process for 5 minutes and separated from the frame by increasing a drawing speed or cutting. As a result, a biodegradable cellulose microneedle containing encapsulated retinol, BSA, and calcein, which was capable of controlling multidrug release, was manufactured to have a top diameter of 5 μm and an effective length of 1,800 μm. The particle size of the emulsion and the microparticles varied according to a stirring speed of the homogenizer and depending on the presence of a filter used, and the length of a manufactured microneedle varied according to a drawing speed and drawing time.

The features and advantages of the present invention will be summarizd as follows:

(i) The present invention provides a method of manufacturing a solid microstructure capable of multidrug release by mixing a biocompatible or biodegradable material with microparticles, nanoparticles, and/or an emulsion as a drug carrier and a solid microstructure manufactured using the same, which have not been adopted in conventional arts.

(ii) According to the present invention, a solid microstructure having a microscale diameter and sufficient effective length and hardness and capable of simultaneously containing a variety of drugs can be manufactured.

(iii) According to the present invention, a solid microstructure can be manufactured as a smart drug containing a mixture of water-soluble and fat-soluble drugs or a mixture of cosmetic ingredients or polymers as a multidrug or capable of controlling multidrug release.

(iv) According to the present invention, a solid microstructure can control the release speed of one drug according to a time or deliver various drugs without pain in an immediate release manner and/or sustained release manner.

It will be apparent to those skilled in the art that various modifications can be made to the above-described exemplary embodiments of the present invention without departing from the spirit or scope of the invention. Thus, it is intended that the present invention covers all such modifications provided they come within the scope of the appended claims and their equivalents.

What is claimed is:

1. A method of manufacturing a solid microstructure for drug release, comprising:
(a) preparing a biocompatible material or biodegradable material as a scaffold material of the solid microstructure;
(b) mixing the biocompatible material or biodegradable material of step (a) with a drug, which is incorporated in microparticles, nanoparticles, or an emulsion; and
(c) manufacturing the solid microstructure containing drug carriers using the mixture of step (b).

2. The method of claim 1, wherein the biocompatible material or the biodegradable material used as the scaffold material of the solid microstructure is one selected from the group consisting of polyester, polyhydroxyalkanoates (PHAs), poly(α-hydroxy acid), poly(β-hydroxy acid), poly(3-hydroxybutyrate-co-valerate) (PHBV), poly(3-hydroxy proprionate) (PHP), poly(3-hydroxyhexanoate) (PHH), poly(4-hydroxy acid), poly(4-hydroxybutyrate), poly(4-hydroxyvalerate), poly(4-hydroxyhexanoate), poly(ester amide), poly(c-caprolactone) (PCL), polylactide (PLA), polyglycolide (PGA), poly(lactide-co-glycolide) (PLGA), polydioxanone, poly(ortho ester), polyetherester, polyanhydride, poly(glycolic acid-co-trimethylene carbonate), polyphosphoester (PPE), PPE urethane, poly(amino acid), polycyanoacrylate, poly(trimethylene carbonate), poly(iminocarbonate), poly(tyrosine carbonate), polycarbonate (PC), poly(tyrosine arylate), polyalkylene oxalate, polyphosphazene, PHA-PEG, polyvinylpyrrolidone (PVP), polybutadiene, polyhydroxy butyric acid, polymethyl methacrylate, polymethacrylic acid ester, polypropylene, polystyrene, polyvinylacetal diethylamino acetate, polyvinyl acetate, polyvinyl alcohol (PVA), polyvinyl butyral, polyvinyl formal, vinyl chloride-propylene-vinyl acetate copolymer, vinyl chloride-vinyl acetate copolymer, coumarone-indene polymer, dibutylamino hydroxypropyl ether, ethylene-vinyl acetate copolymer, glycerol distearate, 2-methyl-5-vinyl pyridine methacrylate-methacrylic acid copolymer, hyaluronic acid, myristic acid, palmitic acid, stearic acid, benenic acid, cellulose or derivatives thereof, maltose, dextran, glucomannan, glucosamine, chitosan, heparin, alginate, inulin, starch, glycogen, chitin, chondroitin, dextrin, keratan sulfate, beef tallow, whale wax, beeswax, paraffin wax, and castor wax.

3. The method of claim 2, wherein the biocompatible material or biodegradable material is PLA, PGA, PLGA, cellulose or derivatives thereof, maltose, dextran, glucomannan, glucosamine, chitosan, heparin, alginate, inulin, starch, or glycogen.

4. The method of claim 1, wherein the microparticles or nanoparticles containing the drug are prepared using one selected from the group consisting of polyester, PHAs, poly(α-hydroxy acid), poly(β-hydroxy acid), PHBV, PHP, PHH, poly(4-hydroxy acid), poly(4-hydroxybutyrate), poly(4-hydroxyvalerate), poly(4-hydroxyhexanoate), poly(ester amide), PCL, PLA, PGA, PLGA, polydioxanone, poly(ortho ester), polyetherester, polyanhydride, poly(glycolic acid-co-trimethylene carbonate), PPE, PPE urethane, poly(amino acid), polycyanoacrylate, poly(trimethylene carbonate), poly(imino carbonate), poly(tyrosine carbonate), PC, poly(tyrosine arylate), polyalkylene oxalate, polyphosphazene, PHA-PEG, PVP, polybutadiene, polyhydroxy butyric acid, polymethyl methacrylate, polymethacrylic acid ester, polypropylene, polystyrene, polyvinylacetal diethylamino acetate, polyvinyl acetate, PVA, polyvinyl butyral, polyvinyl formal, vinyl chloride-propylene-vinyl acetate copolymer, vinyl chloride-vinyl acetate copolymer, coumarone-indene polymer, dibutylamino hydroxypropyl ether, ethylene-vinyl acetate copolymer, glycerol distearate, 2-methyl-5-vinyl pyridine methacrylate-methacrylic acid copolymer, hyaluronic acid, myristic acid, palmitic acid, stearic acid, benenic acid, cellulose or derivatives thereof, maltose, dextran, glucomannan, glucosamine, chitosan, heparin, alginate, inulin, starch, glycogen, chitin, chondroitin, dextrin, keratan sulfate, beef tallow, whale wax, beeswax, paraffin wax, and castor wax.

5. The method of claim 1, wherein the biocompatible material or biodegradable material used as the scaffold material of the solid microstructure is a different material from the microparticles or nanoparticles.

6. The method of claim 1, wherein the solid microstructure contains both the microparticles or nanoparticles and the emulsion as the drug carriers.

7. The method of claim 1, wherein the biocompatible material or biodegradable material used as the scaffold material of the solid microstructure further contains a drug.

8. The method of claim 6, wherein a drug contained in the microparticles or nanoparticles and a drug included in the emulsion are released at different speeds.

9. The method of claim 7, wherein a drug included in the scaffold material of the solid microstructure and a drug included in the microparticles, nanoparticles or emulsion are released at different speeds.

10. The method of claim 6, wherein the scaffold material of the solid microstructure, the microparticles or nanoparticles, and the emulsion contain different kinds of drugs, respectively.

11. The method of claim 1, wherein the microstructure is one selected from the group consisting of a microneedle, a microspike, a microblade, a microknife, a microfiber, a microprobe, a microbarb, a microarray, and a microelectrode.

12. A solid microstructure for drug release comprising a biocompatible material or biodegradable material as a scaffold material thereof, wherein at least one drug carrier selected from the group consisting of microparticles, nanoparticles and an emulsion containing a drug is incorporated into the biocompatible material or biodegradable material.

13. The solid microstructure of claim 12, wherein the biocompatible material or the biodegradable material as the scaffold material of the solid microstructure is one selected from the group consisting of polyester, polyhydroxyalkanoates (PHAs), poly(α-hydroxy acid), poly(β-hydroxy acid), poly(3-hydroxybutyrate-co-valerate) (PHBV), poly(3-hydroxy proprionate) (PHP), poly(3-hydroxyhexanoate) (PHH), poly(4-hydroxy acid), poly(4-hydroxybutyrate), poly(4-hydroxyvalerate), poly(4-hydroxyhexanoate), poly(ester amide), poly(ε-caprolactone) (PCL), polylactide (PLA), polyglycolide (PGA), poly(lactide-co-glycolide) (PLGA), polydioxanone, poly(ortho ester), polyetherester, polyanhydride, poly(glycolic acid-co-trimethylene carbonate), polyphosphoester (PPE), PPE urethane, poly(amino acid), polycyanoacrylate, poly(trimethylene carbonate), poly(iminocarbonate), poly(tyrosine carbonate), polycarbonate (PC), poly(tyrosine arylate), polyalkylene oxalate, polyphosphazene, PHA-PEG, polyvinylpyrrolidone (PVP), polybutadiene, polyhydroxy butyric acid, polymethyl methacrylate, polymethacrylic acid ester, polypropylene, polystyrene, polyvinylacetal diethylamino acetate, polyvinyl acetate, polyvinyl alcohol (PVA), polyvinyl butyral, polyvinyl formal, vinyl chloride-propylene-vinyl acetate copolymer, vinyl chloride-vinyl acetate copolymer, coumarone-indene polymer, dibutylamino hydroxypropyl ether, ethylene-vinyl acetate copolymer, glycerol distearate, 2-methyl-5-vinyl pyridine methacrylate-methacrylic acid copolymer, hyaluronic acid, myristic acid, palmitic acid, stearic acid, benenic acid, cellulose or derivatives thereof, maltose, dextran, glucomannan, glucosamine, chitosan, heparin, alginate, inulin, starch, glycogen, chitin, chondroitin, dextrin, keratan sulfate, beef tallow, whale wax, beeswax, paraffin wax, and castor wax.

14. The solid microstructure of claim 13, wherein the biocompatible material or biodegradable material is PLA, PGA, PLGA, cellulose or derivatives thereof, maltose, dextran, glucomannan, glucosamine, chitosan, heparin, alginate, inulin, starch, or glycogen.

15. The solid microstructure of claim 12, wherein the microparticles or nanoparticles containing the drug are prepared using one selected from the group consisting of polyester, polyhydroxyalkanoates (PHAs), poly(α-hydroxy acid), poly(β-hydroxy acid), poly(3-hydroxybutyrate-co-valerate) (PHBV), poly(3-hydroxy proprionate) (PHP), poly(3-hydroxyhexanoate) (PHH), poly(4-hydroxy acid), poly(4-hydroxybutyrate), poly(4-hydroxyvalerate), poly(4-hydroxyhexanoate), poly(ester amide), poly(c-caprolactone) (PCL), polylactide (PLA), polyglycolide (PGA), poly(lactide-co-glycolide) (PLGA), polydioxanone, poly(ortho ester), polyetherester, polyanhydride, poly(glycolic acid-co-trimethylene carbonate), polyphosphoester (PPE), PPE urethane, poly(amino acid), polycyanoacrylate, poly(trimethylene carbonate), poly(iminocarbonate), poly(tyrosine carbonate), polycarbonate (PC), poly(tyrosine arylate), polyalkylene oxalate, polyphosphazene, PHA-PEG, polyvinylpyrrolidone (PVP), polybutadiene, polyhydroxy butyric acid, polymethyl methacrylate, polymethacrylic acid ester, polypropylene, polystyrene, polyvinylacetal diethylamino acetate, polyvinyl acetate, polyvinyl alcohol (PVA), polyvinyl butyral, polyvinyl formal, vinyl chloride-propylene-vinyl acetate copolymer, vinyl chloride-vinyl acetate copolymer, coumarone-indene polymer, dibutylamino hydroxypropyl ether, ethylene-vinyl acetate copolymer, glycerol distearate, 2-methyl-5-vinyl pyridine methacrylate-methacrylic acid copolymer, hyaluronic acid, myristic acid, palmitic acid, stearic acid, benenic acid, cellulose or derivatives thereof, maltose, dextran, glucomannan, glucosamine, chitosan, heparin, alginate, inulin, starch, glycogen, chitin, chondroitin, dextrin, keratan sulfate, beef tallow, whale wax, beeswax, paraffin wax, and castor wax.

16. The solid microstructure of claim 12, wherein the biocompatible material or biodegradable material used as the scaffold material of the solid microstructure is a different material from the microparticles or nanoparticles.

17. The solid microstructure of claim 12, wherein the solid microstructure contains both the microparticles or nanoparticles and the emulsion as the drug carriers.

18. The solid microstructure of claim 12, wherein the biocompatible material or biodegradable material used as the scaffold material of the solid microstructure further contains a drug.

19. The solid microstructure of claim 17, wherein a drug contained in the microparticles or nanoparticles and a drug included in the emulsion are released at different speeds.

20. The solid microstructure of claim 18, wherein a drug included in the scaffold material of the solid microstructure and a drug included in the microparticles, nanoparticles or emulsion are released at different speeds.

21. The solid microstructure of claim 17, wherein the scaffold material of the solid microstructure, the microparticles or nanoparticles, and the emulsion contain different kinds of drugs, respectively.

22. A solid microstructure prepared using the method of claim 1.
